(19) United States
(12) Patent Application Publication
(10) Pub. No.: US 2015/0024505 A1
(43) Pub. Date: Jan. 22, 2015

(54) APPARATUS FOR EVALUATING CARBONATE SOLUBILITY

(71) Applicant: SAUDI ARABIAN OIL COMPANY, DHAHRAN (SA)

(72) Inventors: Jack D. LYNN, Magnolia, TX (US); Victor V. HILAB, Dhahran (SA)

(21) Appl. No.: 14/322,198

(22) Filed: Jul. 2, 2014

Related U.S. Application Data
(60) Provisional application No. 61/847,259, filed on Jul. 17, 2013.

(51) Int. Cl. G01N 33/24

(52) U.S. Cl. CPC G01N 33/24
USPC 436/100; 422/68.1

(57) ABSTRACT

A method and apparatus of determining solubility of compounds at non-ambient conditions is disclosed. The compound of interest is placed in a reaction vessel which can withstand the non-ambient conditions, and is allowed to react with an acid at those conditions for a pre-determined time. The reaction is quickened thereby stopping the reaction, after which the conditions are permitted to return to ambient and the solubility of the compound is then determined gravimetrically.

### RELATED APPLICATIONS

This application claims priority from U.S. Provisional Application No. 61/847,259 filed Jul. 17, 2013, incorporated by reference in its entirety.

### FIELD OF THE INVENTION

The invention relates to a method and apparatus useful in determining the solubility of a substance, such as a carbonate, at a fixed set of conditions which are non-ambient, i.e., up to a combination of a pressure of 3000 psig and a temperature of 150° C. (wherein “ambient conditions” refers to conditions of 25-30° C., (68-77° F.), and pressures of 14-15 psig). It is particularly useful in determining solubility of the substance in solutions of strong acids.

### BACKGROUND AND PRIOR ART

The dissolution of carbonates (e.g., CaCO₃) at atmospheric conditions is controlled by thermodynamic constraints, including temperature, and the concentration of acid used. In the case of high pressure reactions, however, a further limit is imposed, i.e., a kinetic control, resulting from the build up of CO₂ in the reaction. Dodds, et al., Industrial and Engineering Chemistry, Vol. 1, No. 1, pg. 92-95 (1956), demonstrated that finite amounts of CO₂ can be dissolved in reaction brines at high temperature, which results in a kinetically controlled dissolution mechanism that slows the reactivity of acid and carbonate.

At pH values below 4, and reaction conditions which include ambient temperature, it has been shown that the rate of calcite dissolution is mass transfer limited, without regard to the nature of the acid medium. In strong acids, such as HCl, calcite dissolves readily in the following irreversible reaction;

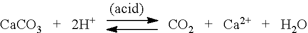

Hence, the dissolution of the calcite is limited by the transport of H⁺ to the calcite surface.

Calcite is the primary component of many minerals, including limestone. Its dissolution can neutralize acidity, increase pH, and the concentration of alkali species, as per:

CaCO₃+2H⁺→Ca²⁺+H₂CO₃* (i)

H₂CO₃+CaCO₃→Ca²⁺+2HCO₃⁻ (ii)

CaCO₃ +H₂O→Ca²⁺HCO₃⁻+OH⁻ (iii)

Note that [H₂CO₃*] is [aqueous CO_{2]}+[H₂CO₃], as per, e.g., Plummer, et al., in “Chemical Modeling In Aqueous Systems,” E. A. Jenne, ed. (American Chemical Society Symposium Series 93: pp. 537-573 (1979)); Stumm, et al., Aquatic Chemistry: Chemical Equilibria and Rates in Natural Waters (Wiley-Interscience, New York) p. 1022 (1996).

The overall rate of dissolution of carbonate containing minerals depends on factors which include the partial pressure of the CO₂ produced, and the activities of H⁺, H₂O, Ca²⁺ and HCO₃⁻. See, e.g., Plummer, et al., supra, and Arakaki, et al., Aquatic Geochemistry 1:105-130 (1995). In general, the rate of calcite dissolution decreases as pH increases, activities of Ca²⁺ and HCO₃⁻ increase, or partial pressure of CO₂ decreases.

All of these guidelines, however, assume that reaction products are removed from reaction media. When the reaction takes place at high pressure, a kinetic control becomes relevant as CO₂ cannot be removed, because its solubility under these conditions drops. As a result, H⁺ generation is blocked, and the pH of the reaction medium increases as reactive acid decreases.

Supersaturation of aqueous media with CO₂ will also cause the reaction to stop and to move toward the left side, i.e., CaCO₃ will no longer dissolve. The reaction cannot continue unless and until a means for removing CO₂ is provided.

Referring to FIG. 1, the solubility of CO₂ in both water and diesel fuel is shown, as a function of pressure and temperature. See, e.g., Dodds, et al., supra.

FIG. 1 shows that the limit of CO₂ solubility is about 150 scf/stb of water (“scf” refers to “standard cubic foot,” “stb” to “stock tank barrel). In practical terms, this is a small amount, as 10% HCl, if allowed to react completely with CaCO₃, will yield this amount of CO₂. At the temperatures and pressures used in petrochemical extraction, a pressure of 10,000 psig, and a temperature of 300° F. are required to reach this level of CO₂ solubility in water. If and when this happens, as noted supra, the reaction stops, and HCO₃⁻ ions are sequested by Ca²⁺, in an intermediate, calcium bicarbonate complex.

The complex is very highly soluble in standard reaction brines, and will not precipitate unless CO₂ is removed. Data show that this complex is possibly a sequestration product for deep sea, i.e., very high pressure, sequestration of CO_{2.} The complex remains stable, if the CO₂ remains in solution. It complexes H₂CO₃ and HCO₃⁻, preventing release of H⁺ from the HCO₃⁻, retarding formation of Ca₂O solutions, and is believed to kinetically limit the reaction with the acid.

Additional evidence suggests that the diffusivity of HCO₃⁻ is reduced at conditions of high temperature and high CO₂ partial pressure. Chizmeshya, et al., “2^{nd} US-China Symposium On CO₂ Emissions Control Science and Technology” (May 28-30, 2008), state that, with temperature as a constant, the diffusivity of HCO₃⁻ will increase as the partial pressure of CO₂ decreases.

One hypothesis from this is that diffusivity of HCO₃ may act as a rate control on the reactivity of CaCO₃ at high CO₂ pressures.

In sum, these data suggest a need to evaluate high temperature reactivity of acid with carbonates when extracting CO₂ from minerals which are under high pressure and high temperature, such as oil and shale reserves. The invention presents an apparatus and method for accomplishing this.

U.S. Pat. No. 7,723,119 discloses a method for determining solubility via using a nearly saturated solution of a compound to be tested, measuring solution pH and then modifying the pH until the compound begins to precipitate from solution. No mention of the use of non-ambient pressure or temperature conditions is found therein.

U.S. Pat. No. 6,521,184, teaches solubility determination via comparing conductivity differentiation between non-decomposed and decomposed compounds.

Russian Patent Application SU 1101297 teaches a device useful for dissolving substances prior to carrying out a reaction. Russian Patent Application Su 1078035 teaches determining solubility of core samples at pre-set conditions, with downhold conditions being simulated. The methods employed in these references use low pressure systems, and do not disclose reactions above ambient pressures.

All references discussed are incorporated by reference herein.

### SUMMARY OF THE INVENTION

The invention is a test device or apparatus useful in determining the absolute solubility of a compound, such as a carbonate, at a specific set of conditions, such as temperature, pressure, and gas saturation, when a specific acid formulation is used, at a single point in time, where the conditions are non-ambient, i.e., where at least one of pressure and temperature vary by at least 10% from the definition, supra. The method, which is also a part of the invention, employs gravimetric analysis of samples.

In operation, the invention allows a reaction, carried out at ambient or non-ambient conditions, to be stopped or quenched, after which the reaction system is brought to ambient temperature and pressure, if necessary, and parameters such as solubility are measured. In effect, a “snapshot” of a reaction is provided.

In action, and as will be elaborated upon, infra, the system design permits bi-directional filing and emptying of a sample cell or vessel, which allows for charging the sample cell with a reactant, such as a strong acid, and then quenching the reaction, under very rapid exchange conditions. In one embodiment, a low temperature chiller loop is employed. This enables the use of quenching materials at a temperature down to about 5° C. This can be critical when the reaction to be quenched generates exothermal heat, which de facto changes reaction conditions, notwithstanding use of a quencher fluid.

In a preferred embodiment, of the invention the apparatus, as discussed and elaborated upon, infra, functions at pressures of up to 2500-3000 psig, and temperatures up to 100-150° C. In practice, it is preferred to test one of temperature and pressure, by keeping one parameter constant and varying the other. This may be seen in, e.g., example 1, infra. The components of the apparatus must be compatible with acids as strong as 20-28% HCl. Nickel-chromium-molybdenum alloys are examples of materials which may be employed in the apparatus of the invention. The method requires heating, which can be managed via, e.g., heating tapes and/or jackets, such as fiber glass jackets, which are manually controlled, or other heating means. In operation, all means for transfer of, e.g., liquids and gases, are heat traced to prevent drops in temperature when fluids are exchanged in, e.g., the reaction vessel. Fluid transfer is always carried out under full test pressure and temperature, and reacted fluids are discharged into heated, pressurized, floating piston accumulators.

The design of the apparatus depicted, and generally, permits bi-directional filling and emptying of the sample cell or vessel in which the reaction takes place, and permits acid and quencher charging in the same configuration, under very rapid conditions. As a result, reacted fluids can be held at reaction conditions, which prevents unwanted precipitation of reactant products. The design of the system permits flushing, so as to separate and to measure ionic loads, entrained gases such as CO₂ and H₂S, and so forth, in the reacted fluids.

### BRIEF DESCRIPTION OF THE FIGURES

FIG. 1 depicts the known limits of CO₂ solubility as a function of pressure.

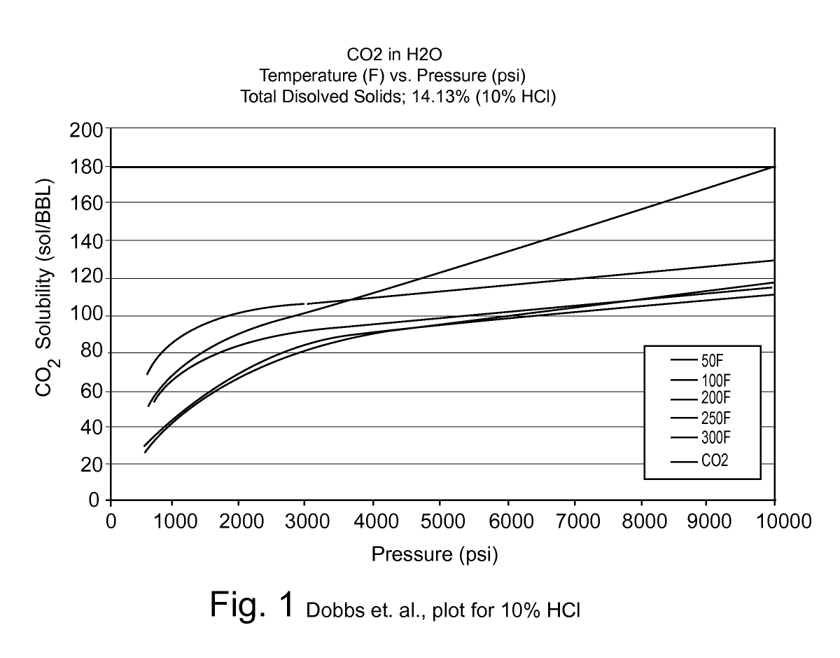

FIG. 2 shows an operating system in accordance with the invention.

FIG. 3 shows the relationship between pressure and temperature arising from experiments using the invention.

FIG. 4 presents results from experiments not carried out in accordance with the invention.

FIG. 5 presents additional data using the invention as a comparison to the results of FIG. 4.

### DETAILED DESCRIPTION OF PREFERRED EMBODIMENTS

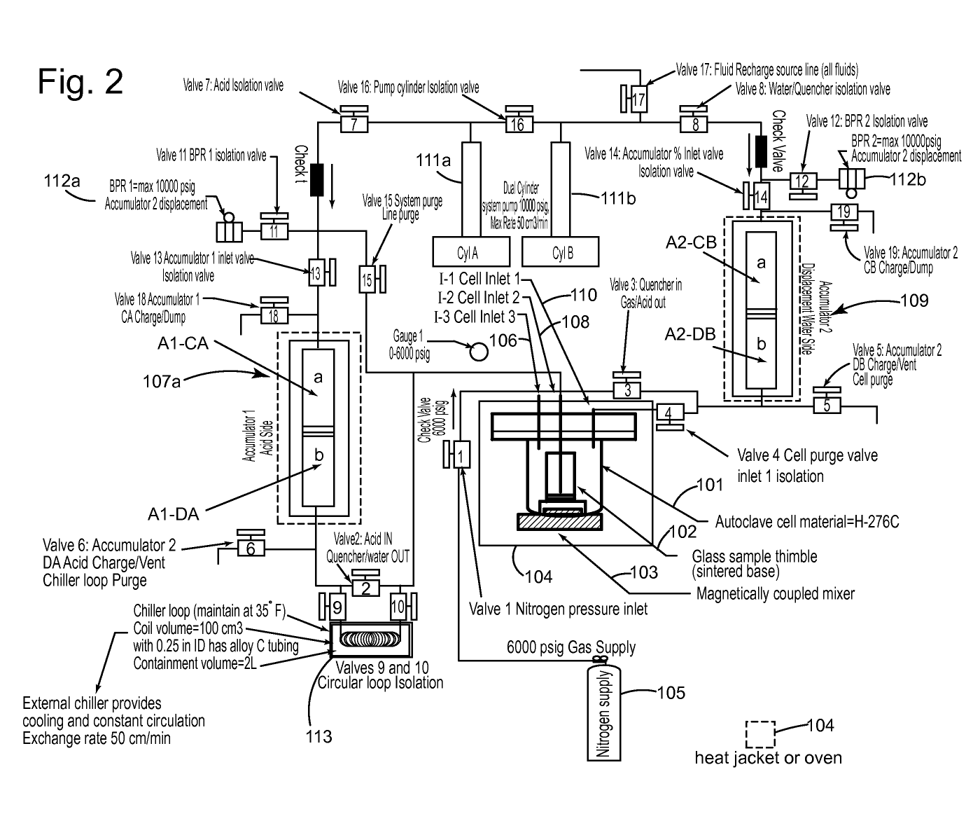

Referring to FIG. 2, one sees one embodiment of the operating system of the invention. A series of valves and other components work in concert. The embodiment shown is manually operated, via physical opening and closing of actuated valves; however, the skilled artisan will envisage other embodiments, such as those which include fully automated valves, and computer programmed test sequences.

Referring to the embodiment of the invention shown in FIG. 2, there are a plurality of valve means, each of which serves a distinct purpose. Valve means (“Vm” hereafter) 1 serves as an inlet valve for increasing nitrogen pressure, from the external N₂ source (105). Vm 3 permits entry of quenching solution into the autoclave cell 101, and passage of gas and acid out of this cell. Vm 5 serves to charge accumulator 109, section 109b, and also to permit venting thereof. Vm 6 permits the input of acid into accumulator 107, section 107b, and displacement of quenching solution and water from the cell, 101. Vm 6 performs several additional functions including venting this section, and acting as a chiller loop purge. Vm 7 and 8 isolate, respectively, acid, and water or quenching solutions. Vm 9 and 10 act to isolate the chilling means represented by chiller loop 113. Vm 11 and 12 act as isolation valves for, respectively BPR-1 and BPR-2 112a and 112b. Vm 13 serves as an inlet valve for section 107a of the first accumulator, with its parallel Vm 14 found for section 109a of the second accumulator. Vm 15 acts to purge system lines. Vm 16 is the pump cylinder isolation valve. Vm 17 controls the fluid recharge source line, for all fluids, and Vms 18 and 19 parallel each other in serving as controlling the charging and emptying of accumulator portions 107a and 109a.

Other features of the device include an autoclave cell 101 which contains a glass sample thimble 102, positioned on a magnetically coupled mixer 103. Seen only at the bottom of the figure, but used to cover the entire apparatus is a heating means, such as a heat jacket or oven 104. A source of nitrogen is seen at 105.

To elaborate, an autoclave cell 101 is provided which contains a glass sample thimble 102, having a sintered base and positioned on a magnetically coupled mixer 103. The entire assembly is contained within heat jacket 104. A source of N₂ 105 having an attached variable pressure control source, allowing pressure from 0-6000 psig is connected to this assembly. N₂ passes through a line and the passage is controlled by Vm 1. N₂ enters the assembly though cell inlet I-3, 106.

Connected to this first assembly is a first accumulator 107, referred to as the “acid accumulator.” This acid accumulator is divided into two parts, 107a and 107b, which will be discussed further, infra. Section 107b of the acid accumulator contains the acid which moves through cell inlet I-2, 108 to provide acid to carry out the analysis of the sample. Vm 2 controls this passage.

Also provided is a second accumulator 109, also covered by a heat jacket referred to as the “water accumulator” hereafter. It, too, is divided into two sections 109a and 109b. Section 109a provides water via means controlled by valve means 14, also directed to the reactor, via inlet I-1, 110. Vm 16 acts to control the purging of liquids from the reaction medium

The following protocol present one approach to using the invention; however, the skilled artisan will recognize that alternate approaches are possible which do not depart from the scope or spirit of the invention.

A sample and the thimble (102) are dried in an oven, for 24 hours at 100° C. After this, they are cooled in a dessicator until testing begins, and the sample weighed. The size of the sample will, of course, depend upon the size of the holding vessel or thimble, but exemplary dimensions are diameters of 0.5-1 inch, and lengths of 1-2 inches. In a preferred embodiment, the sample weighs at least 40 g.

The sample is placed in the thimble, and the combination is weighed. Both this value and the weight of the sample per se are recorded. The sample and thimble are then stored, in desiccators, until ready for testing.

Concurrently, if not prepared previously, reaction solutions are prepared. Distilled water (e.g., 18 mg-ohm water) is combined with HCl, to prepare one or more solutions ranging from 1-28% HCl, e.g., 20% HCl. If desired, additives such as, but not limited to, surfactants, corrosion inhibitors, organic solvents, mutual solvents, and/or scale control agents, may be used. A quenching solution is also prepared, by combining 38.998 g NaOH in one liter of the water described supra, to yield one liter of 1M NaOH solution.

Once the reagents have been prepared, one performs a pretest system set up, and sets the initial valve settings. To elaborate, the system is prepared for an experiment, and all valves are closed. A pair of positive displacement pumps, such as ISCO pumps 111a and 111b, are filled, in both cylinders. The displacement pumps are preferably self-contained, independently controlled entities, with automated fill and delivery valves, as well as delivery and pressure controls. These pumps provide for constant rate and pressure delivery modes of operations, at a pressure of 3000 psig, although the particular settings will vary, based upon the type of dual piston pump used.

Once the pretest system set up is complete, and the initial valve settings are established, a dead liquid side pressure check is performed, by charging cylinders 107 and 109 with distilled water.

To do this, a source of a gas at low pressure (<20 psig) is attached to portions 107a and 109a, respectively, via valves 18 and 19. Vm 5 and 6 are opened, and this allows the pressure in stages 107a and 109A, to drive an internal puck to the bottom of the chamber portion, to provide for a 100% water charge. The source of the gas is removed, and the chambers are depressurized. Vms 5 and 6 are closed.

Following depressurization, the tops of the accumulators 107 and 109 are removed, and the chamber portions are filled with distilled water, than resealed.

The dual cylinder pump means is set at a constant pressure (e.g., 500 psig), while back pressure regulators BPR-1 112a and BPR-2 112b are also set to constant pressures close to that of the dual cylinder pump (e.g., 480 psig).

The dual piston pumps are then started. Pump cylinders are pressurized, and checked for leaks, and valves 7, 8, 11-14, and 17 are opened. Once the pressure in the accumulator exceeds that of the BPR, water is displaced through the BPR, pressurizing the system. Valves 11 and 12 are closed, and the volume displaced by the pumps is monitored for 10 minutes. No change in volume indicates no leak in the tested system.

All valves are then closed, and the system is pressurized to 500 psig. The pumps are turned off, valves 11 and 12 are reopened, and BPRs are checked to make sure they hold the original, constant pressure. Once this is confirmed, valves 13 and 14 are closed. Following this, a sample is loaded, the cell is sealed, and a live liquid side pressure test is carried out.

The sample containing thimble is then placed in the appropriate position in the reactor cell, and the annulus between the cell and thimble is filled with clean, glass beads, so as to provide an inert material which reduces reactor volume. Glass wool is used to cover the beads, and then the cover of the reactor cell is put in place, making sure that the inlet dip line is in the thimble.

The vessel is sealed, as mentioned, by using appropriate means, such as a torsion wrench.

An acid accumulator line is then connected, from accumulator chamber 107b, through Vm 2 and a quencher accumulator line is attached from chamber 109b, through valve 3 to inlet line I-2 (108).

A gas source bottle is then connected, via inlet line I-3, through valve 1, and a quencher side accumulator is connected from 109b through Vm 4 to inlet line I-1.

Valves 9 and 10, which connect to chiller loop means 113, are closed, and then system purge line valve 15 is opened.

The autoclave cell is now checked for leaks, by closing all valves in the system, pressurizing the gas cylinder to 450 psig, opening valve 1, and waiting for the pressure to stabilize in the autoclave cell.

The rest of the experimental system is then checked by making sure only valve 1 is opened to the gas tank, after which valves 2-4 are opened, and the system is checked for leaks. All valves are closed if no leaks are found.

A charge of 400 cm³ of 20% HCl is sent to accumulator 107b, via valve 6. Valves 6, 11, and 13 are opened to allow the charging of the accumulator 107b at 480 psig, to displace the distilled water contained in it, through BPR-1. Following charging, valve 6 is closed, valves 7, 8, 16, and 17 are opened, the pumps are started, with pressure maintenance set at 480 psig. The fitting on Vm 2 is slightly open and acid is bled to valve 2.

After this, BPR-1 112a and -2 112b are set to a given test pressure (e.g., 1100 psig) to test the pressure of the system, and the dual piston pumps are started, so as to increase dead side pressure to 1100 psig. The pumps are started and fluid is displaced through BPR-1 and -2. Once this aspect of the procedure is set in place, valve 8 is closed. The system is now ready for heating, and heating jackets and controllers are placed around accumulator 107. The autoclave cell valve 1 is opened, the gas pressure in the cell is set to 1100 psig, and after the pressure stabilizes, valve 1 is closed.

At this point, the system is ready to initiate heating. BPR-1 112a and -2 112b are set at 1100 psig, accumulator 107a is charged with acid solution at 1100 psig, and accumulator 109 is fully charged with distilled water, at 1100 psig.

The supply of nitrogen is set at a pressure of 1100 psig, and heating jackets are placed around the two accumulators, and the autoclave cell.

Valves 3 and 15 are opened. This is critical for pressure control during heating, and the dual pump pressure is set at 1100 psig. Controllers are set at the required test temperature (within the rage of the system limits) (e.g., 180° F.), the heating system is started, and a mag stirrer is turned on.

The N₂ charge valve 6 is closed when pressure stabilizes, and temperature is kept constant, at test temperature.

The positive displacement pumps are set to their full constant rate.

Accumulator 107b is opened to the test cell via valve 2, and BPR-1 is closed by closing valve 11, while BPR-2 is kept opened via opening valve 12.

The displacement pumps are started, and acid is displaced into the autoclave cell, while any gas in the displacement cell is pushed to 109b. The water in 109a is displaced via BPR-2. The complete volume of 107b is thus displaced through Vm2, through connection 108, and fills cell 101 with reactant acid, at test pressure and temperature. All valves are then closed.

Vm 11 and 15 are opened, to allow for the possibility of slight overpressure during the reaction. Since the BPR's are set at test pressure (e.g., 1100 psig) any overpressure generated in the cell will bleed off through BPR-1 112a.

While the reaction is proceeding, accumulator 107b is cleared, and charged with 500 cm³ of quencher solution, and the chiller turned on with temperature set at 35° F., and full circulation through the chiller bath, 113.

All lines are then bled, and accumulator 110b is cleaned out, via distilled water. During this time, the reactor cell is closed for a pre-set period of time. After this, valves 9, and 10 on the chiller loop, 3 are opened and Vm 11 and 15 are closed. The pumps are started at maximum rate, with valves 7 and 13 open dispensing quencher fluid into the reaction cell at its maximum rate. Simultaneously, fluid is displaced from 107b, and 10a, via BPR-2 112b. All 500 cm³ of the quencher is displaced into, and through the reaction cell. As the quenching reaction is highly exothermic acid stage, this stage passes through the chiller bath, so as to limit reaction heat.

Once this takes place, the valves leading to the cell are shut, and Vm 15 is opened and one waits for 20 minutes, with pressure maintained at 1100 psig.

During this wait time, accumulator 107b is cleaned and filled with 900 cm³ of distilled water. Accumulator 109b is cleaned out, keeping in mind that it contains acid and CO₂ under high pressure. A bleed tray containing Na₂CO₃ is provided to help neutralize the acid. The cleaning is facilitated via distilled water.

After the 20 minute wait time, quencher in the reaction cell is displaced via accumulator 107b and then into accumulator 109b. All 900 cm³ are displaced.

Following displacement, the heaters are turned off and the system is cooled for 30 minutes.

The gas charge valve is opened, and water is purged from the cells via valve means 2 and the receiver vent.

After the displacement of the fluid, the sample and thimble are removed, dried under vacuum for 24 hours, and the sample final weight is taken.

Summarizing the invention, the solubility of a sample is determined gravimetrically, under conditions of pressure and temperature which correspond to the conditions where the substance is found, and to be solubilized. The conditions are produced and regulated as discussed herein, and maintained for a desired time period. Preferably, the time period ranges from about 1 hour to about 12 hours. Conditions may range from ambient, e.g., 14.7 psig of pressure at room temperature, and up to about 3000 psig and about 150° C. At these extreme conditions, it is preferred to maintain the reaction for no more than 12 hours.

After the desired period of time, the reaction is quenched via adding sufficient material to neutralize reactive acid, displacement of the liquid, and washing. Preferably, but not necessarily, the temperature of the reaction is reduced while quenching occurs (because, as noted, supra, the quenching reaction is highly exothermic), followed by reduction in pressure. This multistep process in effect “freezes” the reaction that took place, so that results for those non can-ambient conditions be secured.

While a detailed apparatus and means for operation are shown, any system which allows for rapid exchange, charging and discharging of fluids may be used. Means for administering a solubilizing agent such as an acid, to a reaction vessel must be provided, as must means for a neutralizing substance, and means for removing all liquid which enter the reaction vessel or chamber. Also required is a means for rapid reduction of the temperature and pressure of the reaction must be provided as well. These may include, e.g., chiller means, and bleed valves, for reducing temperature and pressure, respectively.

### EXAMPLE 1

The practice of the invention may be seen in the experiments which follow.

A set of six, randomly selected marble samples were tested to determine the consistency of the data. A 20% HCl solution was used. The samples averaged 98.9 weight % solubility±0.49 weight percent, when tested at 1 atmosphere, and 20° C., after 12 hours of reaction time.

Additional samples were cut from marble standards, and samples were taken from actual carbonate hydrocarbon producing formations as well. Marble plug samples were drilled, from bulk, solid pieces of marble, 1.84 cm in diameter, and 4.78 cm long. Their average weight was 33 g.

Table 1, which follows, shows the results of all of the runs and is followed by a second presentation of the data to show samples broken down into temperature, pressure, and time while in the reaction cell prior to quenching to differentiate the parameters involved.

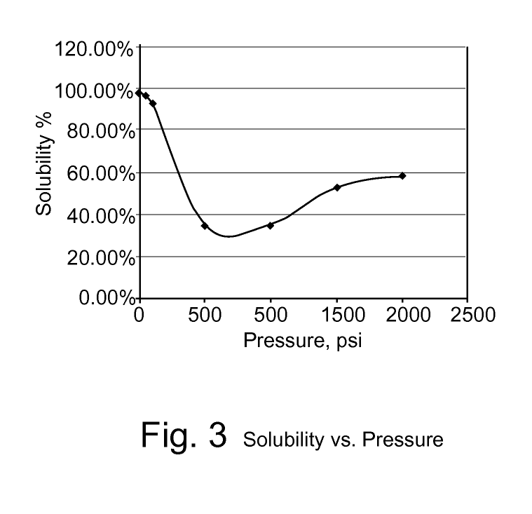

FIG. 3 shows graphically the relationship between pressure and temperature from these experiments.

| Sample set 1 | | | | | Data Summary |
|---|---|---|---|---|---|
| | Parameter | temper- | | Cell | After test |
| Sample | under | ature, | Pressure, | closure | % |
| # | Investigation | ° F. | psig | time, hr | solubility |
| 1 | Pressure | 180 | 0 | 2 | 98.37% |
| 2 | Pressure | 180 | 50 | 2 | 96.59% |
| 3 | Pressure | 180 | 100 | 2 | 92.93% |
| 4 | Closure Time | 180 | 1000 | 12 | 44.50% |
| 5 | Closure Time | 180 | 1000 | 4 | 40.53% |
| 6 | Pressure/ | 180 | 1000 | 2 | 34.07% |
| | Closure Time/ | | | | |
| | temperature | | | | |
| 7 | Closure Time | 180 | 1000 | 8 | 43.55% |
| 8 | Pressure | 180 | 500 | 2 | 34.35% |
| 9 | Closure Time | 180 | 1000 | 6 | 34.50% |
| 10 | Temperature | 210 | 1000 | 2 | 12.61% |
| 11 | Temperature | 240 | 1000 | 2 | 29.30% |
| 12 | Temperature | 270 | 1000 | 2 | 53.77% |
| 13 | Temperature | 300 | 1000 | 2 | 66.37% |
| | Pressure | 180 | 0 | 2 | 98.37% |
| | Pressure | 180 | 50 | 2 | 96.59% |
| | Pressure | 180 | 100 | 2 | 92.93% |
| | Pressure | 180 | 500 | 2 | 34.35% |
| | Pressure | 180 | 1000 | 2 | 34.07% |
| | Pressure | 180 | 1500 | 2 | 51.63% |
| | Pressure | 180 | 2000 | 2 | 57.23% |
| | Temperature | 180 | 1000 | 2 | 34.07% |
| | Temperature | 210 | 1000 | 2 | 33.66% |
| | Temperature | 240 | 1000 | 2 | 37.11% |
| | Temperature | 270 | 1000 | 2 | 44.49% |
| | Temperature | 300 | 1000 | 2 | 66.37% |
| | Closure Time | 180 | 1000 | 2 | 34.07% |
| | Closure Time | 180 | 1000 | 4 | 40.53% |
| | Closure Time | 180 | 1000 | 6 | 34.50% |
| | Closure Time | 180 | 1000 | 8 | 43.55% |
| | Closure Time | 180 | 1000 | 12 | 44.50% |

### EXAMPLE 2

The apparatus of the invention was used to evaluate laboratory results which had shown that carbonates from deep wells, at high temperatures, had reduced solubility in acid.

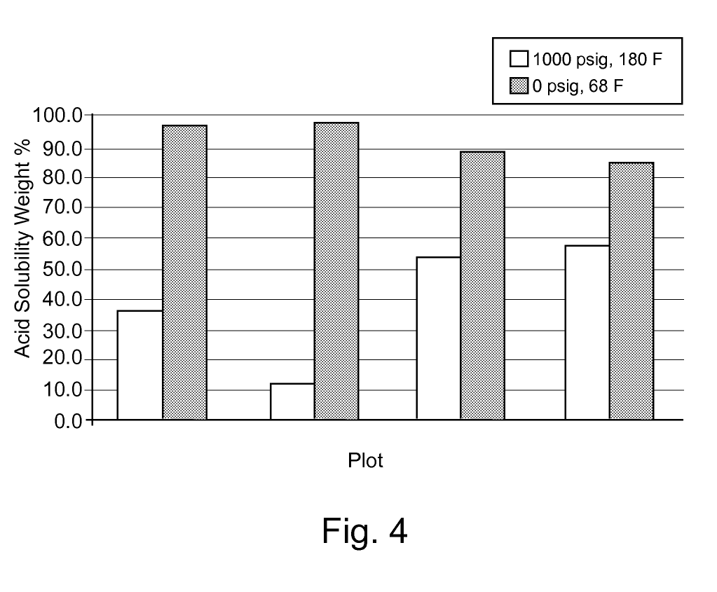

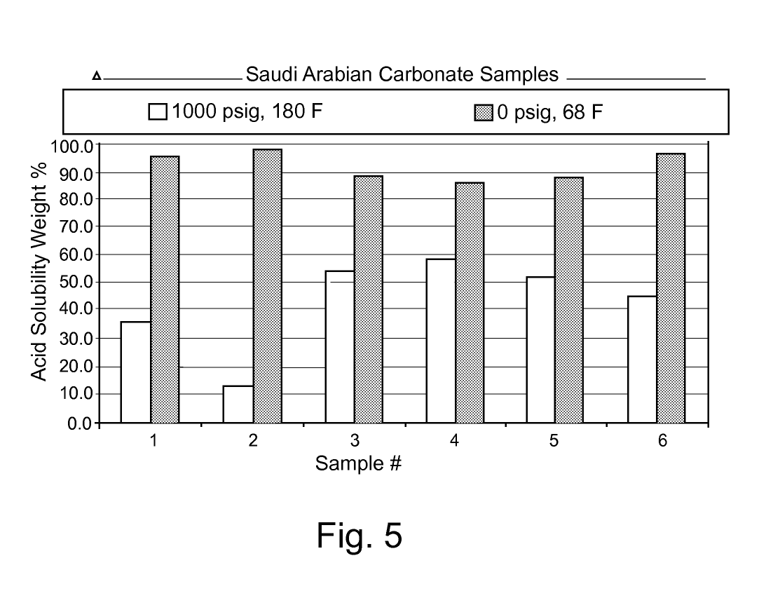

Six samples were tested from a deep well reservoir (Saudi Arabian KIDN reservoir) where previous tests had shown reduced solubility for the carbonates at 1000 psig and 180° F., as compared to ambient conditions. The original results of those experiments are shown in FIG. 4, and contradict conventional wisdom, which is that the solubility of carbonate in HCl should increase as temperatures increase. Yet comparing FIG. 4 to FIG. 5, which compares the results taken with samples of marble, described supra, demonstrate that the reaction is not so simply explained, and the test system is in fact valid.

Other features of the invention will be clear to the skilled artisan and need not be reiterate d here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

We claim:

1. A method for gravimetric determination of solubility of a compound at non-ambient conditions, comprising:
(a) combining said compound with an acid solution in a reaction vessel at conditions of temperature and pressure which are at non-ambient conditions, for a predetermined time in a reaction vessel;
(b) adding a quenching solution to neutralize said acid solution;
(c) displacing said acid solution and said quenching solution from said reaction vessel;
(d) adding a washing solution to said reaction vessel;
(e) removing said washing solution;
(f) adjusting at least one of pressure and temperature in said reaction vessel to ambient conditions, wherein (a), (c), (d), (e), and (f) stop solubilization of said compound, and
(g) gravimetrically determining solubility of said compound at said non-ambient conditions.

2. The method of claim 1, wherein said compound is a carbonate.

3. The method of claim 2, wherein said carbonate is calcium carbonate.

4. The method of claim 1, wherein said acid is HCl.

5. The method of claim 1, wherein said non-ambient conditions comprise one of a pressure of from 2500 to 3000 psig, and a temperature of from 100° C. to 150° C.

6. The method of claim 5, wherein said pressure ranges from 2500-3000 psig.

7. The method of claim 1, wherein said temperature ranges from 100° C. to 150° C.

8. The method of claim 1, wherein said acid solution ranges from 20% to 28% acid.

9. The method of claim 1, comprising reducing said temperature to 5° C.

10. The method of claim 1, further comprising measuring a reaction product produced when solubilizing said compound.

11. The method of claim 10, wherein said reaction product is CO₂ or H₂S.

12. The method of claim 1, wherein said pre-determined time is from 1 hour to 12 hours.

13. Apparatus useful for determining solubility of a compound at non-ambient conditions comprising:
(a) a reaction vessel for reception of said compound and a solvent;
(b) pressuring means for maintaining a desired pressure in said reaction vessel;
(c) at least one delivery means for delivery of solutions to said reaction vessel;
(d) at least one temperature regulating means for regulating temperature of said reaction vessel;
(e) means for removing any solutions provided by (c), and
(f) means for regulating pressure provided by (b).

14. The apparatus of claim 11, wherein (d) comprising a chiller means.

15. The apparatus of claim 11, wherein (f) comprises at least one bleeder valve.
